(19) United States
(12) Patent Application Publication
(10) Pub. No.: US 2022/0331269 A1
(43) Pub. Date: Oct. 20, 2022

(54) COMPOSITIONS FOR TREATING ACNE

(71) Applicants: Botanix Pharmaceuticals Ltd., Northbridge (AU); Botanix Pharmaceuticals, Inc., Plymouth Meeting, PA (US)

(72) Inventors: Eugene R. Cooper, Berwyn, PA (US); Matthew Callahan, Philadelphia, PA (US); Michael Thurn, Kangaroobie (AU); David Dickason, Aston, PA (US)

(21) Appl. No.: 17/856,781

(22) Filed: Jul. 1, 2022

Related U.S. Application Data
(63) Continuation of application No. 16/486,323, filed on Aug. 15, 2019, now abandoned.
(60) Provisional application No. 62/459,312, filed on Feb. 15, 2017.

(30) Foreign Application Priority Data
Feb. 15, 2017 (AU) 2017900498

(51) Int. Cl. A61K 31/08; A61P 17/10; A61K 9/00; A61K 9/08; A61K 47/10; A61K 47/24

(52) U.S. Cl. CPC A61K 31/08; A61P 17/10; A61K 9/0014; A61K 9/08; A61K 47/10; A61K 47/24

(57) ABSTRACT

A topical pharmaceutical composition comprising a solution of 2-(2-ethoxyethoxy)ethanol in a first solvent that is a 2-(2-ethoxyethoxy)ethanol solvent.

### CROSS REFERENCE TO RELATED APPLICATION

This application is a continuation under 35 U.S.C. § 120 to U.S. application Ser. No. 16/486,323, filed Aug. 15, 2019, which is a national phase application under 35 U.S.C. § 371 of PCT International Application No. PCT/AU2018/050117, filed Feb. 14, 2018, which claims the benefit of priority under 35 U.S.C. § 119(e) to U.S. Application No. 62/459,312, filed Feb. 15, 2017 and the benefit of Australian Patent Application No. 2017900498, filed Feb. 15, 2017, the disclosures of which are incorporated herein in their entirety.

### TECHNICAL FIELD

The present invention relates to a pharmaceutical composition for the delivery of 2-(2-ethoxyethoxy)ethanol. The pharmaceutical composition of the present invention is particularly suited for the treatment of acne.

### BACKGROUND ART

The following discussion of the background art is intended to facilitate an understanding of the present invention only. The discussion is not an acknowledgement or admission that any of the material referred to is or was part of the common general knowledge as at the priority date of the application.

Most mammalian skin, including human skin, comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer.

Acne is a multi-factorial disease affecting the sebaceous follicle and characterized by papules, pustules, and scars. Acne affects more than 80% of 16-year old boys and girls, but is not a problem confined to teenagers. Simple attention to hygiene is no longer sufficient and antiseptic washes, so popular some years ago, are now perceived as ineffective by many sufferers and most clinicians.

During puberty, elevated androgen levels stimulate the sebaceous glands to enlarge and produce increased amounts of sebum in the sebaceous follicle. Subsequent abnormal keratinization with hyperkeratosis of the follicular epithelium leads to obstruction of the duct by horny plaque. The blocked duct becomes clogged with a dense material composed of sebum and keratinous debris forming a micro comedo, a precursor of the acne lesion. The excess sebum in the micro comedo also provides an anaerobic growth medium for Propionibacterium acnes. Lipase from the bacteria hydrolyzes sebum triglycerides into free fatty acids that are both comedogenic and pro-inflammatory. Propionibacterium acnes also secrete chemotactic factors that attract neutrophils. Lysosomal enzymes released from the neutrophils rupture the follicle wall releasing pro-inflammatory mediators, including keratin and lipids, into the surrounding dermis. Inflammatory papules appear as a result. Further inflammation with macrophages and foreign body reactions lead to cysts and nodules. The key features of the pathogenesis of acne can be characterized as: 1) increased sebum production; 2) hyper-proliferation of sebocytes (highly specialized, sebum-producing epithelial cells) that contributes to clogging of pores through which sebum is normally released to the skin surface; 3) bacterial proliferation; and 4) inflammation.

Effective management of acne can be accomplished by addressing the four key features of the pathogenesis. Topical therapy is usually the first choice for patients with mild-to-moderate inflammatory acne. The use of topical therapy minimizes potential side effects associated with the use of systemic agents. Topical therapies include benzoyl peroxide, which is the most commonly used non-prescription acne medication. It is an important antibacterial oxidizing agent that can decrease the number of Propionibacterium acnes bacteria and frequently the amount of free fatty acids. Benzoyl peroxide is the first line of monotherapy for mild acne and it is available in over-the-counter preparations. Benzoyl peroxide is applied once or twice daily and patients often experience mild redness and scaling of the skin during the first week of usage.

Tretinoin is an effective topical comedolytic agent, decreasing the cohesiveness of follicular epithelial cells and thereby inhibiting the formation of microcomedones and increasing cell turnover resulting in expulsion of existing comedones. This agent also decreases the thickness of the stratum corneum and potentiates the penetration of topical antibiotic agents. Tretinoin therapy comprises once daily application. Mild redness and peeling are a part of the therapeutic effect of the medication but can result in reduced patient compliance. Patients should be made aware that improvement may take as long as 6 to 12 weeks, and that flare-ups of acne can occur during the first few weeks of therapy. In addition, it is extremely important that patients avoid excessive exposure to the sun during treatment and comply with the designated monitoring program to deal with the well-known side effects of tretinoins.

Mild inflammatory acne lesions can also be treated with topical antibiotics including erythromycin ointment, clindamycin solution and meclocycline cream. The primary action of the antibiotics is to reduce the population of Propionibacterium acnes in the sebaceous follicle and thereby suppress the free fatty acid production. The effectiveness of topical antibiotics in the treatment of acne is limited by their low lipid solubility and subsequent difficulty in penetrating sebum-filled follicles. Topical antibiotics are applied twice daily.

Patients with moderate to severe inflammatory acne often require oral antibiotics in addition to topical therapy. The most commonly prescribed agents include tetracycline, erythromycin, minocycline and doxycycline. Treatment is usually maintained for several months. Side effects include the overgrowth of nonsusceptible organisms including Candida, which can produce vaginal and oral yeast infections.

Patients with severe inflammatory acne unresponsive to other therapy may require treatment with oral isotretinoin. Isotretinoin is a compound related to vitamin A, and is the only agent that decreases sebum production and reverses the abnormal epithelial formation process. This agent can also decrease the population of Propionibacterium acnes in the sebaceous follicle. Duration of therapy is usually 20 weeks and the satisfactory response rate is quite high. However, treatment is often accompanied by many side effects, including dry skin, pruritus, epistaxis and photosensitivity, as well as hypertriglyceridemia, abnormal liver function tests, electrolyte imbalances and elevated platelet counts. Most serious though, is the teratogenic effect of isotretinoin. Use of isotretinoin during pregnancy is absolutely contraindicated. So serious is the potential for death or teratogenic effects to a foetus, isotretinoin is practically contraindicated in women of child-bearing age. Use of isotretinoin must be accompanied by a guarantee by the patient that conception will be avoided at any and all costs.

Because acne is a multi-factorial disease which is manifest to varying degrees, it is important for the physician to assess the patient to attempt to find therapies which will be helpful to the patient without causing major side effects. All of the current conventional treatments are associated with some degree of adverse side effects that limit their usefulness.

### SUMMARY

The present invention provides a topical pharmaceutical composition comprising a solution of 2-(2-ethoxyethoxy)ethanol in a first solvent that is a 2-(2-ethoxyethoxy)ethanol solvent. Preferably the first solvent is siloxane.

The present invention further provides for the use of 2-(2-ethoxyethoxy)ethanol and a first solvent for the manufacture of a topical pharmaceutical composition according to the present invention for the prevention or treatment of acne in a patient in need thereof. Preferably the first solvent is siloxane.

The present invention further provides 2-(2-ethoxyethoxy)ethanol for use in the topical treatment or prevention of acne.

The present invention further provides a combination of 2-(2-ethoxyethoxy)ethanol and a first solvent for use in the topical treatment or prevention of acne. Preferably the first solvent is siloxane.

The present invention further provides a topical pharmaceutical composition comprising 2-(2-ethoxyethoxy)ethanol and a first solvent that is a 2-(2-ethoxyethoxy)ethanol solvent for the treatment or prevention of acne. Preferably the first solvent is siloxane.

The present invention further provides a topical pharmaceutical composition comprising 2-(2-ethoxyethoxy)ethanol, and one or more pharmaceutically acceptable carrier, adjuvants, or vehicles, wherein the pharmaceutical composition does not contain a non-2-(2-ethoxyethoxy)ethanol active pharmaceutical ingredient.

The present invention further provides a method for treating or preventing acne in a patient in need of such treatment, the method comprising topically administering a prophylactically or therapeutically effective amount of a pharmaceutical composition according to the present invention.

### DETAILED DESCRIPTION

2-(2-ethoxyethoxy)ethanol (CAS #111-90-0; also known as diethylene glycol monoethyl ether) is commercially available under the tradenames Transcutol®, among others. 2-(2-ethoxyethoxy)ethanol is listed on the FDA's Inactive Ingredient (IIG) list. Transcutol has been used extensively in other pharmaceutical products and in cosmetics, primarily as a solvent.

However, it has been noted by the inventors that when 2-(2-ethoxyethoxy)ethanol is tested against an active ingredient (for example dapsone gel), it appears to have activity to reduce the number of inflammatory lesions in patient with acne. Without being bound to any theory, we believe that the mechanism for this action is the 2-(2-ethoxyethoxy)ethanol may fluidize the lipids on the skin, thereby retarding the formation of microcomedones. If 2-(2-ethoxyethoxy)ethanol keep lipids from “gluing together” the desquamated keratinocytes, the number of clogged follicles, and subsequently the number of inflammatory lesions, would potentially be reduced. 2-(2-ethoxyethoxy)ethanol in the compositions of the present invention provides a means for stopping the formation of acne at its source; lipids and desquamated keratinocytes clogging pores leading to inflammation and formation of comedones. The compositions of the present invention may be active in clearing acne if used as a routine cleanser.

2-(2-ethoxyethoxy)ethanol is lipophilic. Thus, it poorly absorbed through the skin. Therefore, the success of administering therapeutically effective quantities of 2-(2-ethoxyethoxy)ethanol to a mammal in need thereof within a reasonable time frame and over a suitable surface area has been substantially limited.

Furthermore, 2-(2-ethoxyethoxy)ethanol is a thick and sticky substance. Therefore, the options to administer therapeutically effective quantities of 2-(2-ethoxyethoxy)ethanol to a mammal in need thereof in a manner which does not cause uncomfortable stickiness on the face and/or undesirable transference to clothes and other surfaces, have been substantially limited.

The present disclosure is based on the surprising discovery that 2-(2-ethoxyethoxy)ethanol can be dissolved to form a pharmaceutical composition for treatment of acne. Such a pharmaceutical composition may be topically applied, after which at least some the siloxane evaporates to concentrate 2-(2-ethoxyethoxy)ethanol in situ, facilitating permeation to the therapeutically relevant regions of the skin (preferably the epidermis and dermal layer) for the treatment of acne. Significantly, the 2-(2-ethoxyethoxy)ethanol, which has previously been considered to be a carrier for anti-acne drugs and certain other topically applied drugs, has a therapeutic benefit for treatment of acne. Also importantly, proper formulation of 2-(2-ethoxyethoxy)ethanol facilities permeation of the 2-(2-ethoxyethoxy)ethanol into skin layers that are relevant for exerting a clinical benefit.

The present invention therefore provides a topical pharmaceutical composition comprising a solution of 2-(2-ethoxyethoxy)ethanol in a first solvent that is a 2-(2-ethoxyethoxy)ethanol solvent.

Preferably the first solvent is siloxane.

Topical administration of a pharmaceutical composition that provides high concentrations of dissolved 2-(2-ethoxyethoxy)ethanol (as opposed to solid or crystalline 2-(2-ethoxyethoxy)ethanol) is expected to be advantageous in terms of enhancing the relevant extent of delivery of 2-(2-ethoxyethoxy)ethanol into the skin, particularly the epidermis (including the epidermal basal layer), with some penetration into the dermis. It is thought that the high concentration of dissolved 2-(2-ethoxyethoxy)ethanol on the outer surface of the skin causes a concentration gradient that enhances penetration of the 2-(2-ethoxyethoxy)ethanol into the skin, particularly the epidermis and the dermis.

The 2-(2-ethoxyethoxy)ethanol delivered by the present invention preferably penetrates into the epidermis of the skin, and most of the 2-(2-ethoxyethoxy)ethanol remains in that layer. Preferably, some further penetrates to the dermis, and little 2-(2-ethoxyethoxy)ethanol penetrates further into the hypodermal layer, to be absorbed systemically. The skin to which the composition is delivered is preferably mammalian skin, more preferably human mammalian skin.

The pharmaceutical compositions may include; (i) further volatile solvents such as low molecular weight alcohols, and/or (ii) less volatile solvents such as fatty alcohols and/or alkyl polypropylene glycol/polyethylene glycol ethers (alkyl PEG/PPG ethers). The less volatile solvent is called the residual solvent as it remains on the skin after evaporation of the siloxane (and the further volatile solvent if it is present). These additional volatile and residual solvents may further enhance the capacity of the compositions to produce concentrated 2-(2-ethoxyethoxy)ethanol solutions in situ, and/or facilitate the delivery of the 2-(2-ethoxyethoxy)ethanol to the epidermis and the dermis for the treatment of acne.

### Composition

In order to achieve local distribution for the treatment of acne, it is advantageous for the majority of the 2-(2-ethoxyethoxy)ethanol to penetrate into the epidermis and preferably remain there, and for some 2-(2-ethoxyethoxy)ethanol to further penetrate to the dermis, but little 2-(2-ethoxyethoxy)ethanol to penetrate further into the hypodermal layer and be absorbed systemically. In such a case, the 2-(2-ethoxyethoxy)ethanol would concentrate in the epidermis, thus maximizing its local effect. Not only does the localized effect increase the potential therapeutic benefit, it potentially lessens the frequency and/or severity of any potential side-effects associated with systemic 2-(2-ethoxyethoxy)ethanol administration, because the amount of active compound circulating in the patient is reduced.

The present invention therefore provides a topical pharmaceutical composition comprising a solution of 2-(2-ethoxyethoxy)ethanol in a first solvent that is a 2-(2-ethoxyethoxy)ethanol solvent. Preferably the first solvent is siloxane.

The present invention further provides a pharmaceutical composition comprising 2-(2-ethoxyethoxy)ethanol and a first solvent wherein the 2-(2-ethoxyethoxy)ethanol is dissolved in the composition. The present invention further provides a pharmaceutical composition comprising 2-(2-ethoxyethoxy)ethanol and a first solvent for the treatment or prevention of acne. Preferably the first solvent is siloxane.

The present invention further provides a pharmaceutical composition comprising 2-(2-ethoxyethoxy)ethanol, and one or more pharmaceutically acceptable carrier, adjuvants, or vehicles, wherein the pharmaceutical composition does not contain a non-2-(2-ethoxyethoxy)ethanol active pharmaceutical ingredient.

In one preferred embodiment, the composition is non-aqueous. In another preferred embodiment, the composition does not comprise a preservative.

The present invention is based at least in part on the surprising discovery that 2-(2-ethoxyethoxy)ethanol can be topically administered as (i) concentrated solutions of 2-(2-ethoxyethoxy)ethanol in a 2-(2-ethoxyethoxy)ethanol solvent, or (ii) suspensions of crystalline 2-(2-ethoxyethoxy)ethanol in concentrated solutions of 2-(2-ethoxyethoxy)ethanol in a 2-(2-ethoxyethoxy)ethanol solvent. When used, the compositions may form a highly concentrated, non-crystalline, thin layer of 2-(2-ethoxyethoxy)ethanol on the skin surface, after partial or complete evaporation of the volatile 2-(2-ethoxyethoxy)ethanol solvent and without crystallization of the 2-(2-ethoxyethoxy)ethanol.

Preferably the 2-(2-ethoxyethoxy)ethanol is topically administered as (i) concentrated solutions of 2-(2-ethoxyethoxy)ethanol in siloxane, or (ii) suspensions of crystalline 2-(2-ethoxyethoxy)ethanol in concentrated solutions of 2-(2-ethoxyethoxy)ethanol in siloxane. When used, the compositions may form a highly concentrated, non-crystalline, thin layer of 2-(2-ethoxyethoxy)ethanol on the skin surface, after partial or complete evaporation of the volatile siloxane and without crystallization of the 2-(2-ethoxyethoxy)ethanol.

By using a volatile solvent such as siloxane, one can achieve much higher, non-crystalline (i.e., in solution), concentrations of 2-(2-ethoxyethoxy)ethanol. The 2-(2-ethoxyethoxy)ethanol can be dissolved in much higher concentrations of the volatile solvent than many other less volatile solvents, and then once applied to the skin and the volatile solvent has evaporated, the 2-(2-ethoxyethoxy)ethanol remains on the skin in high concentrations.

The compositions of the present invention can therefore comprise:
2-(2-ethoxyethoxy)ethanol and a first solvent that is a 2-(2-ethoxyethoxy)ethanol solvent;
2-(2-ethoxyethoxy)ethanol, a first solvent that is a 2-(2-ethoxyethoxy)ethanol solvent and a volatile second solvent;
2-(2-ethoxyethoxy)ethanol, a first solvent that is a 2-(2-ethoxyethoxy)ethanol solvent and a third solvent that is less volatile than the first solvent;
2-(2-ethoxyethoxy)ethanol, a first solvent, a volatile second solvent and a third solvent that is less volatile than the first solvent.

Preferably: the first solvent is a siloxane; the volatile second solvent is an alcohol, preferably a low molecular weight alcohol; and the third solvent that is less volatile than the first solvent is a residual solvent, preferably alkyl PEG/PPG ethers and/or fatty alcohols.

### Residual Solvent

The 2-(2-ethoxyethoxy)ethanol is preferably kept in a non-crystalline form on the skin after evaporation of the volatile solvent such as siloxane, by the addition of a less volatile solvent than siloxane. Throughout this specification, this less volatile solvent is called the residual solvent, as it preferably remains on the skin after evaporation of the volatile solvent (for example siloxane and optionally another volatile solvent such as a low molecular weight alcohol) to keep the 2-(2-ethoxyethoxy)ethanol in a non-crystalline state after evaporation of the volatile solvent. Preferably the residual solvent is an alkyl polypropylene glycol/polyethylene glycol ether, and/or a fatty acid alcohol. Preferably the residual solvent has a low volatility such that less than 20% would evaporate at skin temperature over 24 hours. Preferably, the residual solvent has a chain structure that has a hydrophobic end and a hydrophilic end. Preferably the residual solvent is a liquid at or below 32° C. Preferably the residual solvent dissolves siloxane. Preferably the residual solvent maintains the 2-(2-ethoxyethoxy)ethanol in non-crystalline form in concentrations of 20% up to 70% 2-(2-ethoxyethoxy)ethanol.

The total amount of the volatile solvent (siloxane and optionally another volatile solvent such as a low molecular weight alcohol) and the residual solvent if present, required is, once the composition is applied to the skin, sufficient to keep the 2-(2-ethoxyethoxy)ethanol non-crystalline at room temperature for between about 3-8 hours.

Such administration preferably results in enhanced delivery of 2-(2-ethoxyethoxy)ethanol to the epidermis and dermis of the skin compared to pure and/or crystalline 2-(2-ethoxyethoxy)ethanol, which is expected to be effective in significantly reducing, and therefore, treating acne in patients in need of such treatment.

In addition to enhanced delivery, the present compositions may allow larger doses of 2-(2-ethoxyethoxy)ethanol to be applied without having to have a thick layer of residue that might be rubbed off or be unacceptable to the user. The topical pharmaceutical compositions described herein allow more rapid delivery of the 2-(2-ethoxyethoxy)ethanol due to the metastable high driving force or supersaturation of the composition. In summary, and without being bound by any theory, it is thought that the high concentration of dissolved 2-(2-ethoxyethoxy)ethanol on the outer surface of the skin causes a concentration gradient that enhances penetration of the 2-(2-ethoxyethoxy)ethanol into the epidermis and dermis.

The residual solvent may be alkyl polypropylene glycol/polyethylene glycol ethers (alkyl PEG/PPG ethers), and/or fatty alcohols.

The preferred ratio of 2-(2-ethoxyethoxy)ethanol to first solvent to residual solvent is selected from the range consisting of (w/w %): between 0.5-20% 2-(2-ethoxyethoxy)ethanol, between 1-99% first solvent and between 0.1-99% residual solvent; between 5-20% 2-(2-ethoxyethoxy)ethanol, between 4-70% first solvent and between 1%-70% residual solvent; between 1-15% 2-(2-ethoxyethoxy)ethanol, between 20-95% first solvent and between 1-15% residual solvent. Preferably the first solvent is a 2-(2-ethoxyethoxy)ethanol solvent, more preferably the first solvent is siloxane.

The preferred ratio of 2-(2-ethoxyethoxy)ethanol to siloxane to residual solvent is selected from the range consisting of (w/w %): between 0.5-20% 2-(2-ethoxyethoxy)ethanol, between 1-99% siloxane and between 0.1-99% residual solvent; between 5-20% 2-(2-ethoxyethoxy)ethanol, between 4-70% siloxane and between 1%-70% residual solvent; between 1-15% 2-(2-ethoxyethoxy)ethanol, between 20-95% siloxane and between 1-15% residual solvent.

### Siloxane

Siloxanes do not burn, sting or have an odour, and thus are highly advantageous for topical application for the treatment of acne. Importantly for the compositions of the present invention, siloxanes, due to their low molecular weight, are highly volatile.

In one embodiment, the siloxane contains two or three silicon atoms. The siloxanes may have between one and eight methyl groups. In one embodiment, the siloxane is selected from the group consisting of: hexamethyldisiloxane, octamethyltrisiloxane and combinations thereof. These are the most volatile siloxanes, and are thus the most advantageous. Preferably the level of volatility of the siloxane is about the same as that of isopropyl alcohol (IPA).

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such a octamethylcyclotetrasiloxane (CAS #556-67-2) or decamethylcyclopentasiloxane (CAS #541-02-6).

In certain embodiments, further improvements in the solubility and crystallinity characteristics of the 2-(2-ethoxyethoxy)ethanol in the first solvent, preferably siloxane, may be achieved by the addition of a further second volatile solvent in the form of an alcohol, including a low molecular weight alcohol.

An improvement in the solubility and crystallinity characteristics of the 2-(2-ethoxyethoxy)ethanol in the first solvent, preferably siloxane, may also be achieved by the addition of a residual solvent, optionally an alkyl PEG/PPG ether and/or a fatty alcohol.

### Alkyl Polypropylene Glycol/Polyethylene Glycol Ethers

In certain embodiments, further improvements in the solubility characteristics of the 2-(2-ethoxyethoxy)ethanol in the first solvent, preferably siloxane, may be achieved by the addition of alkyl polypropylene glycol/polyethylene glycol ethers (alkyl PEG/PPG ethers). The properties of alkyl PEG/PPG ethers, as well as suitable alkyl PEG/PPG ethers that can be used in accordance with this invention, are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf; accessed 21 Dec. 2016) and the contents of that document are incorporated herein.

The alkyl PEG/PPG ethers may act as a residual solvent to assist in maintaining the 2-(2-ethoxyethoxy)ethanol in a non-crystalline state after evaporation of some or all of the first solvent, preferably siloxane, and the optional low molecular weight alcohol.

Advantageously, in some embodiments, the composition also comprises one or more alkyl PEG/PPG ethers. Alkyl PEG/PPG ethers are the reaction products of an alkyl alcohol and one or more equivalents each of ethylene oxide and propylene oxide (forming repeats of polyethylene glycol (PEG) and polypropylene glycol (PPG), respectively).

The addition of alkyl PEG/PPG ethers, including polypropylene glycol ethers of stearyl alcohol and butyl alcohol, can improve the solubility of 2-(2-ethoxyethoxy) ethanol in siloxane solvents. This ability to increase the concentration of the 2-(2-ethoxyethoxy)ethanol in the initial composition and in the final composition on the skin after application and evaporation makes it possible to achieve high residual concentrations of 2-(2-ethoxyethoxy)ethanol on the skin. The alkyl PEG/PPG ethers may provide a residual solvent that can retain the 2-(2-ethoxyethoxy)ethanol in solution at an exceptionally high concentration after evaporation of the volatile solvent or solvent mixture.

Advantageously, in some embodiments, the alkyl PEG/PPG ethers are liquids at ambient temperatures. Preferably the alkyl PEG/PPG ethers are liquids at about 30° C., or less, or at about 25° C.

Advantageously, in some embodiments, the alkyl PEG/PPG ethers have a low volatility such that less than 20% would evaporate at skin temperature over 24 hours.

Advantageously, in some embodiments, the alkyl PEG/PPG ether has a PEG/PPG chain length of between 10-50 PG units and an ether component of between 2-20 carbons, wherein the sum of the PG units and the carbons of the ether component is preferably between 20 and 60. A range of alkyl PEG/PPG ethers are discussed in the Cosmetic Ingredient Review (CIR) Expert Panel 2013 “Safety Assessment of Alkyl PEG/PPG Ethers as Used in Cosmetics” Report (www.cir-safety.org/sites/default/files/PEGPPG062013tent.pdf; accessed 21 Dec. 2016) and the contents of that document are incorporated herein.

Advantageously, in some embodiments, the alkyl PEG/PPG ether is selected from the group consisting of: polypropylene glycol ethers of stearyl alcohol or butyl alcohol and combinations thereof.

In specific embodiments, the alkyl PEG/PPG stearyl ether or butyl ether is selected from the group consisting of: polypropylene glycol (PPG) stearyl ethers and polypropylene glycol butyl ethers such as PPG-15 stearyl ether and PPG-40 butyl ether and combinations thereof.

In specific embodiments, the relative amount of alkyl PEG/PPG ether is selected from the following group; at least 1% w/w, at least 2% w/w, at least 3% w/w, at least 4% w/w, at least 5% w/w. In specific embodiments, the maximum concentration of the alkyl PEG/PPG ether is 50% w/w. In specific embodiments, the maximum concentration of the alkyl PEG/PPG ether is 80% w/w.

Preferably the amount of alkyl PEG/PPG ether is sufficient to keep the 2-(2-ethoxyethoxy)ethanol is a non-crystalline form on the skin after partial or complete evaporation of the more volatile solvent or solvents.

### Fatty Alcohol

Advantageously, in certain embodiments, the topical composition is further characterised in that the composition comprises a fatty alcohol. The purpose of the fatty alcohol is to act as a residual solvent for the 2-(2-ethoxyethoxy)ethanol once the more volatile components, such as the first solvent, preferably siloxane, and, optionally, the low molecular weight alcohol, have evaporated. In specific embodiments the fatty alcohol is a C₁₂-2₂ fatty alcohol. In specific embodiments, the fatty alcohol is a C₁₆-2₂ fatty alcohol. In specific embodiments, the fatty alcohol is selected from the group consisting of: oleyl alcohol, isostearyl alcohol, octyldodecyl alcohol, 2-hexyl decyl alcohol.

In specific embodiments, the relative amount of fatty alcohol is selected from the following group; at least 2% w/w, at least 3% w/w, at least 4% w/w, at least 5% w/w. In specific embodiments, the maximum concentration of the fatty alcohol is 50% w/w. In specific embodiments, the maximum concentration of the fatty alcohol is 80% w/w.

Preferably the amount of fatty alcohol is sufficient to keep the 2-(2-ethoxyethoxy)ethanol is a non-crystalline form on the skin after partial or complete evaporation of the more volatile solvent or solvents.

### Low Molecular Weight Alcohol

Advantageously, in some embodiments, the topical composition also comprises a second solvent, preferably an alcohol, more preferably a low molecular weight alcohol. The inventors have found that small amounts of a low molecular weight alcohol may improve the solubility of 2-(2-ethoxyethoxy)ethanol in the first solvent, preferably siloxane. This ability to increase the concentration of the 2-(2-ethoxyethoxy)ethanol in the initial composition makes it possible to achieve high residual concentrations of 2-(2-ethoxyethoxy)ethanol on the skin after application. Preferably the low molecular weight alcohol forms a further volatile solvent in addition to the first solvent, preferably siloxane. Preferably the level of volatility of the low molecular weight alcohol is about the same as that of isopropyl alcohol. The addition of a second volatile solvent such as a low molecular weight alcohol may be of particular advantage if the concentration of 2-(2-ethoxyethoxy)ethanol in the initial composition is very high.

Advantageously, in some embodiments, the low molecular weight alcohol is a liquid at ambient temperatures. Preferably the low molecular weight alcohol is liquid at about 30° C., or less, or at about 25° C. Preferably the level of volatility of the low molecular weight alcohol is about the same as that of isopropyl alcohol (IPA). Preferably, the boiling point of the low molecular weight alcohol is less than about 100° C. at standard pressure. Preferably, the boiling point of the low molecular weight alcohol is less than about 90° C. at standard pressure. Preferably, the boiling point of the low molecular weight alcohol is less than about 85° C. between about 80 and about 85° C. at standard pressure.

Advantageously, in some embodiments, the low molecular weight alcohol is selected from the group consisting of: C₂₋₆ alcohols, and combinations thereof. Advantageously, in some embodiments, the low molecular weight alcohol is selected from the group consisting of: C₂₋₄ alcohols, and combinations thereof.

In specific embodiments, the low molecular weight alcohol is selected from the group consisting of: ethyl alcohol (or ethanol), n-propanol, isopropyl alcohol, butanol and combinations thereof.

In specific embodiments, the relative amount of low molecular weight alcohol is selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w. In specific embodiments, the maximum concentration of the low molecular weight alcohol is 50% w/w. In specific embodiments, the maximum concentration of the low molecular weight alcohol is 60% w/w, 70% w/w, 80% w/w. The amount of low molecular weight alcohol may be between 1% w/w and 50% w/w, 1% w/w and 40%, 1% w/w and 30% w/w, 1% w/w and 20% w/w, 1% w/w and 10% w/w.

2-(2-ethoxyethoxy)ethanol

In certain embodiments, the concentration of 2-(2-ethoxyethoxy)ethanol in the topical composition of the invention may be selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, and at least 15% w/w.

In certain embodiments, the concentration of 2-(2-ethoxyethoxy)ethanol in the topical composition may be selected from the group consisting of: at least 20% w/w, at least 30% w/w at least 40% w/w, at least 50% w/w, at least 60% w/w, at least 65% w/w, at least 70% w/w, at least 80% w/w, at least 90% w/w, at least 95% w/w and at least 99% w/w. Such concentrations may be achieved after at least partial evaporation of the volatile siloxane and, optionally, low molecular weight alcohol components.

In certain embodiments, the concentration of 2-(2-ethoxyethoxy)ethanol in the topical composition may be within a range with a lower limit selected from the group consisting of: 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, and 15% w/w;

and an upper limit selected from the group consisting of:
20% w/w, 30% w/w, 40% w/w, 50% w/w, 60% w/w, 65% w/w, 70% w/w, 80% w/w, 90% w/w, 95% w/w, and 99% w/w.

In certain embodiments, the concentration of the 2-(2-ethoxyethoxy)ethanol in the topical composition may be within a range selected from the group consisting of:

1% w/w, 2% w/w to 99% w/w, 3% w/w to 70% w/w, 4% w/w to 70% w/w, 5% w/w to 70% w/w, 6% w/w to 70% w/w, 7% w/w to 70% w/w, 8% w/w to 99% w/w, 9% w/w to 99% w/w, 10% w/w to 99% w/w, 11% w/w to 99% w/w, 12% w/w to 99% w/w, 13% w/w to 99% w/w, 14% w/w to 99% w/w, and 15% w/w to 99% w/w.

In certain embodiments, the concentration of the 2-(2-ethoxyethoxy)ethanol in the topical composition may be within a range selected from the group consisting of:

1% w/w, 2% w/w to 95% w/w, 3% w/w to 95% w/w, 4% w/w to 95% w/w, 5% w/w to 95% w/w, 6% w/w to 95% w/w, 7% w/w to 95% w/w, 8% w/w to 95% w/w, 9% w/w to 95% w/w, 10% w/w to 95% w/w, 11% w/w to 95% w/w, 12% w/w to 95% w/w, 13% w/w to 95% w/w, 14% w/w to 95% w/w, and 15% w/w to 95% w/w.

In certain embodiments, the concentration of the 2-(2-ethoxyethoxy)ethanol in the topical composition may be within a range selected from the group consisting of:

1% w/w, 2% w/w to 90% w/w, 3% w/w to 90% w/w, 4% w/w to 90% w/w, 5% w/w to 90% w/w, 6% w/w to 90% w/w, 7% w/w to 90% w/w, 8% w/w to 90% w/w, 9% w/w to 90% w/w, 10% w/w to 90% w/w, 11% w/w to 90% w/w, 12% w/w to 90% w/w, 13% w/w to 90% w/w, 14% w/w to 90% w/w, and 15% w/w to 90% w/w.

In certain embodiments, the concentration of the 2-(2-ethoxyethoxy)ethanol in the topical composition may be within a range selected from the group consisting of:

1% w/w, 2% w/w to 80% w/w, 3% w/w to 80% w/w, 4% w/w to 80% w/w, 5% w/w to 80% w/w, 6% w/w to 80% w/w, 7% w/w to 80% w/w, 8% w/w to 80% w/w, 9% w/w to 80% w/w, 10% w/w to 80% w/w, 11% w/w to 80% w/w, 12% w/w to 80% w/w, 13% w/w to 80% w/w, 14% w/w to 80% w/w, and 15% w/w to 80% w/w.

In certain embodiments, the concentration of the 2-(2-ethoxyethoxy)ethanol in the topical composition may be within a range selected from the group consisting of:

1% w/w, 2% w/w to 70% w/w, 3% w/w to 70% w/w, 4% w/w to 70% w/w, 5% w/w to 70% w/w, 6% w/w to 70% w/w, 7% w/w to 70% w/w, 8% w/w to 70% w/w, 9% w/w to 70% w/w, 10% w/w to 70% w/w, 11% w/w to 70% w/w, 12% w/w to 70% w/w, 13% w/w to 70% w/w, 14% w/w to 70% w/w, and 15% w/w to 70% w/w.

In certain embodiments, the concentration of the 2-(2-ethoxyethoxy)ethanol in the topical composition may be within a range selected from the group consisting of:

1% w/w, 2% w/w to 65% w/w, 3% w/w to 65% w/w, 4% w/w to 65% w/w, 5% w/w to 65% w/w, 6% w/w to 65% w/w, 7% w/w to 65% w/w, 8% w/w to 65% w/w, 9% w/w to 65% w/w, 10% w/w to 65% w/w, 11% w/w to 65% w/w, 12% w/w to 65% w/w, 13% w/w to 65% w/w, 14% w/w to 65% w/w, and 15% w/w to 65% w/w.

In certain embodiments, the concentration of the 2-(2-ethoxyethoxy)ethanol in the topical composition may be within a range selected from the group consisting of:

1% w/w, 2% w/w to 60% w/w, 3% w/w to 60% w/w, 4% w/w to 60% w/w, 5% w/w to 60% w/w, 6% w/w to 60% w/w, 7% w/w to 60% w/w, 8% w/w to 60% w/w, 9% w/w to 60% w/w, 10% w/w to 60% w/w, 11% w/w to 60% w/w, 12% w/w to 60% w/w, 13% w/w to 60% w/w, 14% w/w to 60% w/w, and 15% w/w to 60% w/w.

In certain embodiments, the concentration of the 2-(2-ethoxyethoxy)ethanol in the topical composition may be within a range selected from the group consisting of:

1% w/w, 2% w/w to 50% w/w, 3% w/w to 50% w/w, 4% w/w to 50% w/w, 5% w/w to 50% w/w, 6% w/w to 50% w/w, 7% w/w to 50% w/w, 8% w/w to 50% w/w, 9% w/w to 50% w/w, 10% w/w to 50% w/w, 11% w/w to 50% w/w, 12% w/w to 50% w/w, 13% w/w to 50% w/w, 14% w/w to 50% w/w, and 15% w/w to 50% w/w.

In certain embodiments, the concentration of the 2-(2-ethoxyethoxy)ethanol in the topical composition may be within a range selected from the group consisting of:

1% w/w, 2% w/w to 40% w/w, 3% w/w to 40% w/w, 4% w/w to 40% w/w, 5% w/w to 40% w/w, 6% w/w to 40% w/w, 7% w/w to 40% w/w, 8% w/w to 40% w/w, 9% w/w to 40% w/w, 10% w/w to 40% w/w, 11% w/w to 40% w/w, 12% w/w to 40% w/w, 13% w/w to 40% w/w, 14% w/w to 40% w/w, and 15% w/w to 40% w/w.

In certain embodiments, the concentration of the 2-(2-ethoxyethoxy)ethanol in the topical composition may be within a range selected from the group consisting of:

1% w/w, 2% w/w to 30% w/w, 3% w/w to 30% w/w, 4% w/w to 30% w/w, 5% w/w to 30% w/w, 6% w/w to 30% w/w, 7% w/w to 30% w/w, 8% w/w to 30% w/w, 9% w/w to 30% w/w, 10% w/w to 30% w/w, 11% w/w to 30% w/w, 12% w/w to 30% w/w, 13% w/w to 30% w/w, 14% w/w to 30% w/w, and 15% w/w to 30% w/w.

In certain embodiments, the concentration of the 2-(2-ethoxyethoxy)ethanol in the topical composition may be within a range selected from the group consisting of:

1% w/w, 2% w/w to 20% w/w, 3% w/w to 20% w/w, 4% w/w to 20% w/w, 5% w/w to 20% w/w, 6% w/w to 20% w/w, 7% w/w to 20% w/w, 8% w/w to 20% w/w, 9% w/w to 20% w/w, 10% w/w to 20% w/w, 11% w/w to 20% w/w, 12% w/w to 20% w/w, 13% w/w to 20% w/w, 14% w/w to 20% w/w, and 15% w/w to 20% w/w.

### Other Agents

The 2-(2-ethoxyethoxy)ethanol could be incorporated into a composition with an additional active moiety that is capable of improving the appearance and/or hydration of the skin. In addition, the composition of the present invention can be used in conjunction with other topically applied analgesic and/or systemically available agents for the treatment of acne.

However, in many cases, the pharmaceutical composition consists or consists essentially of 2-(2-ethoxyethoxy)ethanol and suitable solvents. Thus, the composition is free of conventional active ingredients for treating acne or other conditions. That is, the only active ingredient in the composition of the invention is 2-(2-ethoxyethoxy)ethanol. Therefore, the present invention provides a pharmaceutical composition comprising 2-(2-ethoxyethoxy)ethanol, and one or more pharmaceutically acceptable carrier, adjuvants, or vehicles, wherein the pharmaceutical composition does not contain a non-2-(2-ethoxyethoxy)ethanol active pharmaceutical ingredient.

Thus, in certain cases, the composition does not contain one or more of: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds that are not present include one or more of: morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

In certain cases the composition does not include one or more of: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

In certain cases, the composition does not include one or more of: xylocaine, cocaine, lidocaine, benzocaine, etc., which may provide a more immediate, if less effective in the long run, level of pain relief until the analgesic agent becomes fully effective.

In certain cases, the composition does not include dextromethorphan.

In certain cases, the pharmaceutical compositions described herein do not include some or all of the following agents for the treatment of acne: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

### Acne Treatment and Therapy

In certain embodiments the topical application of 2-(2-ethoxyethoxy)ethanol by way of the compositions described herein is expected to reduce the incidence and/or severity of acne. Therapeutic effects of the present invention include, but are not limited to, reduction in redness, reduction in oiliness, itch, pain or irritation, a reduction in pimples, papules, blisters or pustules, a reduction in infection, a reduction of swelling, cracking, weeping, crusting, and scaling and/or a general decrease in inflammation.

In certain embodiments, the topical application of 2-(2-ethoxyethoxy)ethanol by way of the compositions described herein is expected to improve the symptoms of acne.

The term “improve” is used to convey that the present invention changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, reduction in oiliness of the skin, decreased spread of blisters, decreased ulceration of the skin, decreased redness, reduction of scarring, reduction in lesions, healing of blisters, reduced skin thickening, closure of wounds and lesions, a reduction in symptoms including, but not limited to, pain, inflammation, itching, milia or other symptoms associated with inflammatory conditions or the like.

Treatment can lead to improved healing of the skin. For example, when used in the treatment of acne, swollen, cracked or scaled skin may heal more quickly and/or completely, compared to when left untreated.

Treatment can result in one or more therapeutic effects. Therapeutic effects in the affected area include, but are not limited to, reduction in redness, itch, pain or irritation, the number and severity of the acne lesions, a reduction in infection, a reduction of swelling, cracking, weeping, crusting, and scaling and/or a general decrease in inflammation. One or more of these therapeutic effects are expected to be observed when treatment in accordance with the present invention is made to any of the suitable conditions.

In one aspect, the present disclosure is directed to methods of treating acne using topical 2-(2-ethoxyethoxy)ethanol. In accordance with certain embodiments, a topical composition containing 2-(2-ethoxyethoxy)ethanol is preferably applied topically to an area which is affected by acne. Preferably, the application of 2-(2-ethoxyethoxy)ethanol in accordance with certain embodiments results in reduction in redness, itch, pain or irritation, a reduction in pimples, papules, blisters or pustules, a reduction in infection, less breakdown and loss of collagen and elastin in the skin, a reduction of swelling, cracking, weeping, crusting, and scaling and/or a general decrease in inflammation.

The present invention therefore provides a method for treating or preventing acne in a patient in need of such treatment, the method comprising topically administering a prophylactically or therapeutically effective amount of a pharmaceutical composition according to the present invention.

The present invention further provides for the use of 2-(2-ethoxyethoxy)ethanol and a first solvent, preferably siloxane, for the manufacture of a topical pharmaceutical composition according to the present invention for the prevention or treatment of acne in a patient in need thereof.

The present invention further provides 2-(2-ethoxyethoxy)ethanol for use in the topical treatment or prevention of acne.

### Pharmaceutical Composition

Certain embodiments of the present composition comprise any topically acceptable non-transdermally effective carrier vehicle. Preferred topically acceptable vehicles include but are not limited to gels, ointments, and liquids. Administration of the preferred embodiment is performed in accordance with that mode which is most amenable to the topically acceptable form chosen. For example, gels, lotions, creams and ointments are preferably administered by spreading.

The dilution of the 2-(2-ethoxyethoxy)ethanol in the topical composition can be an important consideration. The 2-(2-ethoxyethoxy)ethanol concentration in the composition should be high enough that the patient does not need to wait an excessively long time for the composition to dry. On the other hand, the 2-(2-ethoxyethoxy)ethanol concentration should be dilute enough that a patient can achieve effective coverage of the affected area. Additionally, the composition could include a component which polymerizes in response to exposure to air or ultraviolet radiation.

The amount of composition to be applied will vary depending on the choice of first solvent (e.g. siloxane), second solvent (e.g. low molecular weight alcohol), residual solvent (e.g. fatty alcohol, and/or alkyl PEG/PPG ether) as well. For example, when the 2-(2-ethoxyethoxy)ethanol is administered by spraying a solution of the drug, the total volume in a single dose may be as low as 0.1 ml. When the 2-(2-ethoxyethoxy)ethanol is administered in a gel or cream, the total volume may be as high as 10 ml. Conversely, if acne comprises scattered lesions, the volume applied to each lesion may be smaller. The carrier selected, and its manner of application, are preferably chosen in consideration of the needs of the patient and the preferences of the administering physician.

In one preferred embodiment, the composition comprises a gel which is preferably administered by spreading the gel onto the affected area. In other preferred embodiments, the composition comprises a liquid, which can be administered by spraying or otherwise applying the liquid onto the affected area. The composition may or may not contain water. Preferably, the composition does not contain water, i.e. it is non-aqueous.

The quantities of the applied 2-(2-ethoxyethoxy)ethanol described herein in the. In general, amounts therapeutically equivalent to 0.1 to 200 mg of 2-(2-ethoxyethoxy)ethanol applied to an area of 5-100 cm², are preferred.

In accordance with certain embodiments, the composition is applied to the affected area regularly until relief is obtained. In one preferred embodiment, the composition is administered to the skin of the patient in need of such treatment using a dosing regimen selected from the group consisting of: every hour, every 2 hours, every 3 hours, once daily, twice daily, three times daily, four times daily, five times daily, once weekly, twice weekly, once fortnighly and once monthly. However, other application schedules may be utilized in accordance with the present invention.

In certain embodiments, the composition of the invention may be provided in a form selected from the group comprising, but not limited to a liquid or gel, a leave-on preparation, a wash-off preparation.

In one embodiment, the composition comprises impurities, wherein the quantity of impurities as a percentage of the total weight of the composition is selected from the group consisting of: less than 20% impurities (by total weight of the composition); less than 15% impurities; less than 10% impurities; less than 8% impurities; less than 5% impurities; less than 4% impurities; less than 3% impurities; less than 2% impurities; less than 1% impurities: less than 0.5% impurities; less than 0.1% impurities. In one embodiment, the composition comprises microbial impurities or secondary metabolites, wherein the quantity of microbial impurities as a percentage of the total weight of the composition is selected from the group consisting of: less than 5%; less than 4%; less than 3%; less than 2%; less than 1% s; less than 0.5%; less than 0.1%; less than 0.01%; less than 0.001%. In one embodiment, the composition is sterile and stored in a sealed and sterile container. In one embodiment, the composition contains no detectable level of microbial contamination.

### Definitions

The following definitions in this specification are intended to be interpreted in an illustrative, rather than limiting sense. Therefore, they are to be interpreted inclusively, and are not to be limited to the specific definition recited.

Antagonist: a compound that does not enhance or stimulate the functional properties of a receptor, yet block those actions by an agonist.

Bandage: a dressing used to cover an afflicted area.

Central nervous system: the brain and spinal cord.

Dermal: relating to the dermis.

Dressing combine: designed to provide warmth and protection to absorb large quantities of fluid that may drain from an incision or wound; consists of a nonwoven fabric cover enclosing fibre with or without absorbent tissue.

Inflammation: an immune system-mediated process characterized by redness, heat, swelling, and pain at the local site.

Mammal: vertebrates with hair, three middle ear bones and mammary glands. Mammals include humans.

Skin: the outer covering of an animal body. Mammalian skin comprises three layers: (i) an epidermis layer, which is predominantly composed of keratinocytes and a small number of melanocytes and Langerhans cells (antigen presenting cells); (ii) a dermis layer, which contains nerve endings, sweat glands and oil (sebaceous) glands, hair follicles, and blood vessels and which is primarily composed of fibroblasts; and (iii) a hypodermis layer of deeper subcutaneous fat and connective tissue. The epidermis itself is made up of two layers, the outer stratum corneum and the inner epidermal basal layer, sometimes referred to as the basement membrane. The purpose of the stratum corneum is to form a barrier to protect underlying tissue from infection, dehydration, chemicals and mechanical stress.

Therapeutically-effective amount: the amount necessary to bring about a therapeutic effect.

Transdermal: passing through the dermis.

### General

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### Example 1

Example Techniques for Ascertaining Permeability of Compositions Containing 2-(2-ethoxyethoxy)ethanol.

The permeability of human skin has been studied for several decades. The skin consists of two major layers, the outer epidermis and the inner dermis. The stratum corneum (“SC”), the outermost 10-20 μm of the epidermis, is responsible for the skin's excellent diffusional resistance to the transdermal delivery of most drugs. Most of the skin's enzymatic activity lies in the basal cell layer of the viable epidermis. Fibrous collagen is the main structural component of the dermis. The skin vasculature is supported by this collagen and lies a few microns underneath the epidermis. Basically, it is here that permeation ends and systemic uptake begins. Many researchers have developed skin permeability relationships based on the physicochemical parameters (molecular weight, molecular volume, lipophilicity, hydrogen-bonding potentials, polarity, etc.) of skin penetrants. However, when dealing with transdermal administration of 2-(2-ethoxyethoxy)ethanol these skin permeability relationships need to be modified to take into account the potential complications of extreme lipophilicity and concurrent metabolism.

Selection and optimization of 2-(2-ethoxyethoxy)ethanol for delivery into the epidermis and dermis requires an understanding of cutaneous metabolism. Furthermore, since skin metabolism of topical in vivo studies cannot easily be distinguished from blood, liver, or other tissue metabolism, cutaneous metabolism is better studied in vitro. However, the success of any such in vitro study depends heavily on finding ideal conditions to simulate in vivo conditions, especially in maintaining tissue viability. Thus, selection of an optimal receiver solution is critical to the success of any such in vitro studies.

A high-pressure liquid chromatography (HPLC) assay can be used for the analysis of 2-(2-ethoxyethoxy)ethanol in samples. An appropriate HPLC system may consist of a Waters 717 plus Autosampler, Waters 1525 Binary HPLC Pump and Waters 2487 Dual A Absorbance Detector with Waters Breeze software. A Brown-lee C-18 reversed-phase Spheri-5 μm column (220×4.6 mm) with a C-18 reversed phase 7 μm guard column (15×3.2 mm) may be used with the UV detector set at a wavelength of 215 nm. The mobile phase may comprise of acetonitrile: 25 mM phosphate buffer with 0.1% triethylamine pH 3.0 (80:20). An appropriate flow rate of the mobile phase would be 1.5 mL and 100 μL of the sample would be injected onto the column.

A PermeGear flow-through (In-Line, Riegelsville, Pa.) diffusion cell system is appropriate for the skin permeation studies. Trans-epidermal water loss can be measured (Evaporimeter EPI™, ServoMed, Sweden) after securing the skin in the cells. Pieces of skin with readings below 10 g/m2/h would be used for the diffusion studies. The skin surface in the diffusion cells would be maintained at 32° C. with a circulating water bath. An appropriate receiver solution would be HEPES-buffered Hanks' balanced salts with gentamicin (to inhibit microbial growth) containing 40% polyethylene glycol 400 (pH 7.4), and the flow rate was adjusted to 1.1 mL/h. An excess quantity of CBD would be added to the donor vehicle (propylene glycol: Hanks' buffer (80:20)) solution with and without permeation enhancers at 6% v/v, sonicated for 10 min, and then applied onto the skin. Excess quantity of the drug would be used in the donor compartment throughout the diffusion experiment in order to maintain maximum and constant chemical potential of the drug in the donor vehicle. Each cell would appropriately be charged with 0.25 mL of the respective drug solution. Samples would appropriately be collected in 6 h increments for 48 h. All the samples would appropriately be stored at 4° C. until HPLC analysis.

Drug disposition in the skin samples would be measured at the completion of the 48 h experiment. The skin tissue would be rinsed with nanopure water and blotted with a paper towel. To remove the drug formulation adhering to the surface, the skin would be tape stripped twice using book tape (Scotch®, 3M, St. Paul, Minn.). The skin in contact with the drug would be excised, minced with a scalpel and placed in a pre-weighed vial. Drug would be extracted from the skin by equilibrating with 10 mL of ACN in a shaking water bath overnight at room temperature. Samples would be analyzed by HPLC to determine CBD content in micromoles (μm) of drug per gram of wet tissue weight. Statistical analysis of the in vitro human skin permeation data could be performed using SigmaStat 2.03. A one-way ANOVA with Tukey post-hoc analysis could be used to test the statistical differences among the different treatments.

### Example 2

A Split-Face Study to Evaluate the Reduction in Skin Surface Oil with BTX1701 Solution Compared to Normal Skin Cleansing in Patients with Oily Skin.

The object of this study is to determine the ability of BTX1701 solution to decrease skin surface oil and shininess in subjects with oily skin as measured through analysis of skin photography.

TABLE 1
BTX1701 Formulation

| Compound | Conc (w/w) |
|---|---|
| Transcutol | 10%-40% |
| Hexylmethyldisiloxane | 40%-90% |
| Alkyl polypropylene | 0-10% |
| glycol PPG-15 | |
| Isopropyl Alcohol | 0-10% |

### Methods

Subjects will begin screening to determine eligibility to participate in the study. Demographics, height, weight, and concomitant medications will be collected. This study included male and female participants who were between 18 and 65 years of age (inclusive). Participants were in good general health without clinically significant disease. Subjects should have oily skin as defined by ≥150 μg/cm2/hr of sebum as measured with a Sebumeter. Subjects should not have sunburns, unevenness in skin tones, tattoos, scars, excessive hair, freckles, birthmarks, moles, or other skin damage or abnormality that would result in the inability to evaluate the skin of the face. Subjects should also not have any skin condition of the face other than oily skin such as, but not limited to, atopic dermatitis, perioral dermatitis, or rosacea. Mild acne is allowed.

A Sebumeter will be used to assess the level of skin oiliness on the forehead. A review of the eligibility criteria will occur.

Within 5 days and no less than 24 hours after the Screening Visit, if the subject is eligible, the Treatment Visit will occur. Prior to any treatment, photographs of the face will be obtained using the Canfield VISIA-CR system. Subjects will alternately have either the right or left side of the face treated with BTX1701 (i.e., left, right, left, right, left). The contralateral side will be cleansed with Cetaphil® Daily Facial Cleanser. Study site staff will apply the BTX1701 and cleanse the face with provided treatment pads.

Following treatment, photographs of the subject's face will be obtained at 30 minutes, and at 1, 2, 3, and 4 hours after treatment.

Cutaneous tolerability assessments will also be obtained at 30 minutes and at 4 hours after treatment. Adverse events will be monitored throughout the 4-hour period after treatment.

After completion of the photography at 4 hours after treatment, the subject will be discharged from the study.

The study site will use the Sebumeter® SM 815 manufactured by Courage+Khazaka Electronic GmbH, Köln, Germany. Prior to measuring the SER, the subjects will first acclimate to the surrounding environment for 30 minutes. The clinical staff will then wipe their foreheads with 70% isopropyl alcohol and allow the area to dry for 5 minutes. After the 1-hour sebum collection period, Sebumeter measurements will be taken. The site will be divided into 3 sections according to a template. One measurement will be taken from each section and the location will be marked on the diagram for that participant and the average measurement recorded.

Subjects will receive their application of investigational product on Day 1 at the clinical site administered by the clinical site staff. Three mL of the investigational product will be dispensed onto a pad. The clinical site staff will then apply the investigational product to one side of the face. Application will alternate from one side of the face to the other for each subject enrolled (i.e., left side for Subject 001, right side for Subject 002, left side for subject 003, etc.). The investigational product should be applied in a consistent manner for each application. The following order of application will be followed:

1. Forehead

2. Cheek to jaw line

3. Nose

4. Upper lip

5. Chin to jaw line

### Safety Assessment

Safety will be assessed through collection of adverse event reports and cutaneous tolerability assessments. All treatment-emergent adverse events (TEAEs) occurring during the study will be recorded listed by subject. Treatment-emergent adverse events are those AEs with an onset on or after the first application of study medication. All reported TEAEs will be summarized by the side of the face treated (BTX1701 or cleanser), the number of subjects reporting events, severity, relationship to investigational product, and seriousness. Serious adverse events (SAEs) will be listed by subject.

Subjects will be monitored for cutaneous tolerability after treatment with the investigational product. Cutaneous tolerability assessments will be conducted by the principal investigator or an appropriately trained designee. Signs and symptoms of cutaneous tolerability will be graded using the following scale:

0, None; 1, Slight; 2, Moderate; 3, Intense.

Evaluation for, and grading of, the following will be done at each evaluation:
Erythema,
Scaling,
Dryness,
Burning/Stinging, and
Irritant/allergic contact dermatitis

### Skin Oiliness, Shininess, and Fluorescence Imaging

From the Baseline photographs, a certified Image Analysis Technician (IAT) will delineate an Area of Interest (AOI) on the Left, Right, and Front View images of a subject. Either all the follow-up images of this subject will be elastically registered with respect to the baseline images, or the Baseline delineated AOI will be registered with all follow-up images of the subject so as to maintain same analysis area across all time-points. During registration, the IAT may adjust the AOI to ensure obstructions and interferences are not included.

A combination of cross and parallel polarized images of the subject for a certain time-point and view will then be analyzed by an automated algorithm for identifying the silvery/white specular, and uniform gloss regions of skin within the AOI. The algorithm will report total area and mean/median intensity measurements for both regions. Measurements obtained from the silvery/white specular regions will be evaluated for shine, while those from the uniform gloss regions will be evaluated for skin surface oiliness. The same AOI will be used for analyzing corresponding time-point and view fluorescence image of the subject. Yellowish-Green fluorescing spots will be detected as Coproporphyrin III fluorescence, while red fluorescing spots will be detected as Protoporphyrin IX fluorescence. The algorithm will report total number of spots, total area, and mean/median intensity for both fluorescence signals. Measurements obtained from yellowish-green fluorescing spots will be evaluated as an indicator of P. acnes bacterial distribution and colonization, while measurements from the red fluorescing spots will be evaluated as an indicator of sebum production.

### Statistical Analysis

All statistical processing will be performed using SAS® unless otherwise stated.

Photographs of the full face will be obtained prior to and at 30 minutes, 1, 2, 3, and 4 hours after treatment. Treatment is defined as application of BTX1701 to one side of the face and cleansing with the facial cleanser to the contralateral side of the face.

At each time point, photographs will be analyzed for shine and oiliness. Silvery/white specular areas of skin will be evaluated as/for shine, while areas with mostly uniform gloss will be evaluated as/for oiliness. Fluorescence images will be analyzed for measurement of green-yellow (Coproporphyrin III) and red (Protoporphyrin IX) fluorescence signal.

Demographics will be summarized by age, gender, race, ethnicity height and weight. For continuous variables, the mean, standard deviation (SD), median, and range will be presented along with the 95% confidence interval (CI). Categorical variables will be summarized by proportions along with the 95% CI.

A sample of 5 subjects is considered adequate to determine a distinction in the ability of the BTX1701 solution to decrease facial oiliness and shininess as assessed by Canfield's specialized photography equipment.

From the foregoing Examples, it is expected that the use of transcutol in accordance with the present invention can be used to treat and/or improve the healing of acne. Generally, treatment in accordance with the present invention will result in a shortened healing time.

1. A topical pharmaceutical composition comprising a solution of 2-(2-ethoxyethoxy)ethanol in a first solvent that is a 2-(2-ethoxyethoxy)ethanol solvent.

2. The pharmaceutical composition of claim 1 wherein the first solvent is a siloxane.

3. The pharmaceutical composition of claim 1 wherein the first solvent:
a) contains 2 or 3 silicon atoms;
b) has a level of volatility about the same as that of isopropyl alcohol; and/or
c) is selected from the group consisting of: hexamethyldisiloxane, octamethyltrisiloxane and combinations thereof.

4. The pharmaceutical composition of claim 1 further comprising a second solvent.

5. The pharmaceutical composition of claim 4 wherein the second solvent is a low molecular weight alcohol.

6. The pharmaceutical composition of claim 1 further comprising a third solvent that is less volatile than the first solvent.

7. The pharmaceutical composition of claim 6 wherein the third solvent is a residual solvent.

8. The pharmaceutical composition of claim 7 wherein the third solvent is a residual solvent chosen from alkyl polypropylene glycol/polyethylene glycol ethers (alkyl PEG/PPG ethers), and/or fatty alcohols.

9. The pharmaceutical composition of claim 1 consisting essentially of 2-(2-ethoxyethoxy)ethanol, the first solvent and a volatile second solvent.

10. The pharmaceutical composition of claim 1 consisting essentially of 2-(2-ethoxyethoxy)ethanol, the first solvent and a third solvent that is less volatile than the first solvent.

11. The pharmaceutical composition of claim 1 consisting essentially of 2-(2-ethoxyethoxy)ethanol, the first solvent, a volatile second solvent and a third solvent that is less volatile than the first solvent.

12. The pharmaceutical composition of claim 8 wherein the alkyl PEG/PPG ether:
a) has a PEG/PPG chain length of between 10-50 PG units and an ether component of between 2-20 carbons, wherein the sum of the PG units and the carbons of the ether component is between 20 and 60;
b) has a low volatility such that less than 5% would evaporate at skin temperature over 24 hours;
c) is a liquid at about 30° C., or less; and/or
d) is selected from the group consisting of: polypropylene glycol ethers of stearyl alcohol and butyl alcohol.

13. The pharmaceutical composition of claim 8 wherein the relative amount of alkyl PEG/PPG ether is:
a) selected from the following group: at least 1% w/w, at least 2% w/w, at least 3% w/w, at least 4% w/w, and at least 5% w/w; and/or
b) a maximum concentration of 50% w/w; or
c) a maximum concentration of 80% w/w.

14. The pharmaceutical composition of claim 8 wherein the fatty alcohol:
a) has a low volatility such that less than 5% would evaporate at skin temperature over 24 hours;
b) is a C12-22 fatty alcohol;
c) is a liquid at about 30° C., or less; and/or
d) is selected from the group consisting of: oleyl alcohol, isostearyl alcohol, octyldodecyl alcohol, and 2-hexyl decyl alcohol.

15. The pharmaceutical composition of claim 8 wherein the relative amount of the fatty alcohol is:
a) selected from the following group: at least 1% w/w, at least 2% w/w, at least 3% w/w, at least 4% w/w, and at least 5% w/w;
b) a maximum concentration of 50% w/w; or
c) a maximum concentration of 80% w/w.

16. The pharmaceutical composition of claim 5 wherein the low molecular weight alcohol:
a) is a liquid at ambient temperatures;
b) has a level of volatility about the same as that of isopropyl alcohol; and/or
c) is selected from the group consisting of: C2-6 alcohols, and combinations thereof; or
d) is selected from the group consisting of: C2-4 alcohols, and combinations thereof; and/or
e) is selected from the group consisting of: ethyl alcohol, n-propanol, isopropyl alcohol and combinations thereof.

17. The pharmaceutical composition of claim 15 wherein the relative amount of low molecular weight alcohol is:
a) selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w
b) a maximum concentration of 50% w/w;
c) a maximum concentration of 60% w/w, 70% w/w, 80% w/w; or
d) between 1% w/w and 50% w/w, 1% w/w and 40%, 1% w/w and 30% w/w, 1% w/w and 20% w/w, 1% w/w and 10% w/w.

18. The pharmaceutical composition according to claim 1 characterised in that the concentration of 2-(2-ethoxyethoxy)ethanol is selected from the group consisting of:
a) at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w and at least 15% w/w; or
b) at least 20% w/w, at least 30% w/w, at least 40% w/w, at least 50% w/w, at least 60% w/w, at least 70% w/w, at least 80% w/w, at least 90% w/w, at least 95% w/w, and at least 99% w/w.

19. A method for treating or preventing acne in a patient in need of such treatment, the method comprising topically administering a prophylactically or therapeutically effective amount of a pharmaceutical composition according to any one of the preceding claims.

20. Use of 2-(2-ethoxyethoxy)ethanol and a first solvent for the manufacture of a pharmaceutical composition according to any one of the preceding claims for the prevention or treatment of acne in a patient in need thereof.

21. A topical pharmaceutical composition comprising 2-(2-ethoxyethoxy)ethanol and a first solvent for the treatment or prevention of acne.

22. The use of claim 20 or composition of claim 21 wherein the first solvent is siloxane.

23. Use of a pharmaceutical composition according to any one of the preceding claims for the prevention or treatment of acne.

24. 2-(2-ethoxyethoxy)ethanol for use in the topical treatment or prevention of acne.

25. A topical pharmaceutical composition comprising 2-(2-ethoxyethoxy)ethanol, and one or more pharmaceutically acceptable carriers, adjuvants, or vehicles, wherein the pharmaceutical composition does not contain a non-2-(2-ethoxyethoxy)ethanol active pharmaceutical ingredient.
